(19) United States
(12) Patent Application Publication
(10) Pub. No.: US 2009/0071470 A1
(43) Pub. Date: Mar. 19, 2009

(54) Emergency medication dose nebulizer

(76) Inventor: Robert Abrams, Oakdale, NY (US)

Correspondence Address: ALFRED M. WALKER, 225 OLD COUNTRY ROAD, MELVILLE, NY, 11747-2712, US

(21) Appl. No.: 11/901,628

(22) Filed: Sep. 18, 2007

(51) Int. Cl. A61M 16/10

(52) U.S. Cl. 128/200.22

(57) ABSTRACT

A conventional respiratory nebulizer has an emergency medication dose storage system conveniently useable in an emergency to deliver the stored medication dose directly to the nebulizing chamber quickly, reliably, and with a single impulse of manual force to a simple mechanical delivery system, thereby making the nebulizer useable in two steps: (a) deploy the medication with a single stroke of force; and (b) inhale the nebulized medication. The nebulizer can be operated without disassembling the nebulizer housing so as to expose the nebulizing chamber and without manually opening the liquid medication container and, without spillage and without manual pouring of the liquid medication directly into the nebulizing chamber, and without reassembling the nebulizer housing before positioning the inhaler mouthpiece in the mouth so as to inhale the nebulized medication.

### FIELD OF THE INVENTION

The present invention relates to a conventional nebulizer having a novel integral structure for conveniently delivering a dose of liquid medication to the conventional nebulizer's conventional nebulizing chamber

### BACKGROUND OF THE INVENTION

Pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started.

The problem is that this series of steps requiring steady hands and manual dexterity may be difficult to achieve for a an asthma attack sufferer who may be panicking because he/she can't breathe pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe.

Pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe. pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe. pulmonary medication may be needed by persons with breathing problems in a hurry. Typically a person experiencing an asthma attack is desperate to get medication. A nebulizer is an air pump device with a small plastic chamber attached to a mouthpiece. Prior art requires the nebulizer to be opened, liquid medication added to the chamber, the chamber closed and the pump started. The problem is that this series of steps requiring steady hands and manual dexterity may be difficult to achieve for a an asthma attack sufferer who may be panicking because he/she can't breathe.

### OBJECTS OF THE INVENTION

It is an object of the present invention to provide a device for quickly and conveniently delivering a dose of liquid medication to the nebulizing chamber of a conventional nebulizer in an emergency.

It is a further object of the invention to provide reliable nebulized medication to a user in an emergency.

It is a further object of the invention to provide emergency nebulized medication to a user where the user is already in acute respiratory distress at the time the user locates the conventional nebulizer and has no person to assist with following the steps required to conventionally nebulize medication, to wit: (1) disassemble the nebulizer housing so as to expose the nebulizing chamber; (2) locate a container of liquid medication to be nebulized; (3) open the liquid medication container, being careful not to spill it; (4) pour the liquid medication directly into the nebulizing chamber without losing any of it through spilling into the nebulizer housing; (5) reassemble the nebulizer housing; and (6) position the inhaler mouthpiece in the mouth so as to inhale the nebulized medication.

It is a further object of the present invention to simplify the conventional procedure required to be followed by the user of a medication nebulizer, which conventional procedure may be critically complex for a person suffering from acute respiratory distress at the time the user locates the conventional nebulizer.

It is a further object of the preferred embodiment of the present invention to provide a simplified reliable process for deploying a dose of liquid medication in a nebulizer, comprising the steps of (1) deploy the medication with a single twist of a screw cap; and (2) inhale the nebulized medication.

It is a further object of the non-preferred embodiment of the present invention to provide a simplified reliable process for deploying a dose of liquid medication in a nebulizer, comprising the steps of (1) deploy the medication with a single stroke of force; and (2) inhale the nebulized medication.

It is a further object of the present invention to provide a novel medication dose delivery device built-in and integrated with a conventional nebulizer to accomplish the result of simplified reliable delivery of the liquid medication to the conventional nebulizing chamber by convenient user deployment without the need to disassemble and reassemble the nebulizer and to open an pour liquid medication at the time of an acute respiratory emergency.

It is a further object of the present invention to provide a conventional nebulizer having a stored single does of liquid medication directly on board and integral with the conventional nebulizer in loaded-gun arrangement in preparation for use in an acute respiratory emergency.

It is a further object of the present invention to reduce the time needed for a person suffering an acute respiratory emergency to receive an effective dose of nebulized medication, particularly where the suffering person has no readily available assistance in using a nebulizer.

It is a further object of the preferred embodiment of the present invention to provide a nebulizer device with a stored dose of liquid medication where deployment of that dose is accomplished by a single twist of a screw cap by a user.

It is a further object of the non-preferred embodiment of the present invention to provide a nebulizer device with a stored dose of liquid medication where deployment of that dose is accomplished by a single stroke of pressure by a user.

It is a further object of the preferred embodiment of the present invention to provide a simplified method of administering a nebulized dose of medication employing the herein provided single-twist screw cap.

It is a further object of the non-preferred embodiment of the present invention to provide a simplified method of administering a nebulized dose of medication employing the herein provided single-pressure stroke.

It is a further object of the present invention to provide a method for speeding relief to sufferers of acute respiratory distress by reducing the time and effort required to deploy liquid medication in a nebulizer.

It is a further object of the present invention to provide reliable faster and simpler relief to a sufferer of acute respiratory distress who is alone and without assistance by reducing the time and effort required to deploy liquid medication in a nebulizer.

In keeping with the present invention other objects will make themselves clear to users of the device and to those of skill in the art, and thus this invention is not limited to the objectives here enumerated, which are not exhaustively presented and are described merely by way of example.

### SUMMARY OF THE INVENTION

In keeping with these objects and others which may become apparent, the present invention relates to a conventional nebulizer having a novel integral structure for conveniently delivering a dose of liquid medication to the conventional nebulizer's conventional nebulizing chamber, the novelty being in providing a new structural component integral with the structure of a conventional nebulizer.

The preferable component is a chamber for vertically mounting the dosage capsule therein from above, to apply torque to squeeze the capsule and unload its content's directly into the misting chamber.

Alternatively, the capsule can be loaded through an external sleeve or chute.

The novel structural component comprises a storage chamber for storing, in loaded-gun fashion, a dose of liquid medication on board the conventional nebulizer housing with a simple user-operable single pressure stroke needed to deploy the medication into the conventional nebulizing chamber. The novel structure medication storage chamber generally has a tapered-nozzle open-aperture delivery end disposed in close proximity to the nebulizing chamber so that the liquid medication, when deployed by a user, is injected reliably and directly into the nebulizing chamber.

The novel medication storage chamber of the non-preferred embodiment accepts a single disposable and user-replaceable cartridge containing a dose of medication to be nebulized in an emergency. The chamber is provided at its outer end with pressure means for a user to exert a stroke of physical force upon the outer end of the medication chamber so as to squeeze upon the medication cartridge.

In the preferred embodiment, the twist off cap handle portion is held in place by a restraining member, while either torque or sideways pressure is applied to the capsule, thereby releasing the seal between the twist off cap and the vessel containing the medication.

In another embodiment, the medication dose cartridge of the non-preferred embodiment can be provided with a seal that is capable of rupture upon hydraulic pressure, the seal being located at an inner end of the cartridge, disposed at or near the inner end of the medication storage chamber. The simplest emergency user-pressure means is a piston arrangement, such as a medical syringe. The non-preferred embodiment of the present invention may be described as a medical syringe capable of receiving disposable medication dose cartridges, in combination with a conventional nebulizer. The medical syringe of the non-preferred embodiment projects through the wall of the conventional nebulizer and has its delivery nozzle at or very near the conventional nebulizing chamber.

The novel combination of medication dose capsule with a conventional nebulizer provided in the present invention, both preferred and non-preferred embodiments, addresses and solves the problem of what procedure must be followed by a patient having a breathing emergency, such as a severe attack of asthma, and needs a quick reliable dose of nebulized medication, particular where (1) no other person is available to assist the patient and (2) a single-shot hand-held nebulizer is medically inappropriate for treatment.

### BRIEF DESCRIPTION OF THE DRAWINGS

The present invention can best be understood in connection with the accompanying drawings. It is noted that the invention is not limited to the precise embodiments shown in drawings, in which: The present invention can best be understood in connection with the accompanying drawings. It is noted that the invention is not limited to the precise embodiments shown in drawings, in which. The present invention can best be understood in connection with the accompanying drawings. It is noted that the invention is not limited to the precise embodiments shown in drawings, in which:

FIG. 1 Shows an exploded view of a prior art nebulizer disassembled to illustrate pouring of medication into the nebulizing chamber;

FIG. 2 shows a user operating a conventional nebulizer by breathing through the mouthpiece;

FIG. 3 shows a perspective view of the non-preferred embodiment having a conventional nebulizer having a novel built-in medication storage chamber extending outward from the housing of the nebulizer;

FIG. 4 shows a non-preferred embodiment medication dose cartridge having an inner end with tapered shoulders so as to be capable of nesting within the medication storage chamber; the medication cartridge has an outer end having means of accepting force for the purpose of ejecting the liquid medication contained in the cartridge through its inner end and into the nebulizing chamber;

FIG. 5 shows a detail of the non-preferred embodiment of the present invention's novel medication storage chamber extending outward from the nebulizing chamber through the wall of the nebulizer housing, having a medication dose cartridge therewithin and having a piston for application of force by a user to break the seal of the medication cartridge. The injection nozzle of the medication storage chamber is shown in close proximity to the nebulizing chamber within the housing;

FIG. 6 shows an exploded view of a first version of the preferred embodiment, having a vertical storage sleeve for a capsule of liquid medication, where the capsule is seated with its tear-off tab in close proximity to the conventional nebulizing chamber within the housing of the conventional nebulizer;

FIG. 7 shows a detailed crossectional view in cutaway of the preferred embodiment of the present invention;

FIG. 8 shows an exploded view of a second version of the preferred embodiment, having a vertical storage sleeve for a capsule of liquid medication, where the capsule is seated with its tear-off tab in close proximity to the conventional nebulizing chamber within the housing of the conventional nebulizer;

FIG. 9 shows a top exploded view of the second version of the preferred embodiment, having a vertical storage sleeve for a capsule of liquid medication, showing a lever moving the capsule laterally, while the tear-off portion of the capsule is seated and immobilized, so that lateral pushing of the capsule causes a tear of the capsule between the tear-off portion and the fluid reservoir portion;

FIG. 10 is a close-up detail crossectional view in cutaway, showing the rotation of the capsule while the tear-off portion is seated immobile in place;

FIG. 11 is a close-up detail bottom view of the sleeve showing the restraining stop means and mist-accommodating ports;

FIG. 12 is an exploded perspective view of an alternate embodiment for a knob cam activation assembly for dispensing medication from a capsule;

FIG. 13 is a bottom view of the knob cam activation assembly shown in FIG. 12;

FIG. 14 is a top plan view of the knob activator thereof;

FIG. 15 is a bottom view of the knob activator as in FIG. 14;

FIG. 16 is a bottom view of the cam assembly shown in FIG. 12.

### LIST OF REFERENCE NUMERALS

10. Nebulizer Housing
11. Connecting Tube between nebulizer housing 10 and breather 25
14. Conventional medication dose container including:
   Nebulizer chamber;
   Compressed air supply line; and,
   Conventional breather portion of conventional nebulizer;
30. Conventional mouthpiece at proximal end of conventional breather 25
31. Open distal end of conventional breather 25.
35. Novel storage chamber for medication dose.
36. Inner end of medication storage chamber 35.
37. Outer end of medication storage chamber 35.
38. Tapered open-ended nozzle at inner end 36 of medication storage chamber 35.
40. User-Removable user-replaceable medication dose cartridge containing a dose of liquid medication to be nebulized.
41. Outer end of Medication dose cartridge 40.
42. Inner end of Medication dose cartridge 40.
43. Pressure seal at inner end 42 of Medication dose cartridge 40.
44. Elastomerically Sealed Piston at outer end 41 of cartridge 40.
45. Open reduced-diameter inner end of Medication dose cartridge 40.
46. Tapered inner shoulders of medication Cartridge 40.
50. Grooved piston rod.
52. Finger engagement wings.
55. Stop for engaging groove of Piston Rod 50
56. Pressure plate at the end of Piston Rod 50 for application of user force.
62. Vertical medication storage sleeve 62.
62a, 62b. Slots in sleeve 62 to allow fluid to enter reservoir 15
62c. Restraining stop means for tear off portion of capsule 66
62d. aperture for fluid flow into reservoir 15
64. Tear off tab.
66. Medication dose capsule.
68. Screw cap activating handle.
69. Activating lever handle.
69a. Activating lever handle rod.
69b. Activating lever handle paddle.
70. Inhaling pipe.
162a. Mist port.
162b. Mist port.
162c. Restraining stop means.
168. Knob activator.
180a. Capsule pincher blade.
180b. Capsule pincher blade.
190. (Cam assembly.
192a. Cam contact element.
192b. Cam contact element.
194.a Rotation stop element.
194b. Reciprocating rotation stop element.

### DETAILED DESCRIPTION OF THE INVENTION

In keeping with the objects of the invention, the present invention provides a conventional nebulizer having a built-in (and thus integral) novel storage structure for storing a dose of liquid medication in preparation for an emergency. The liquid medication is conveniently delivered to the conventional nebulizer's conventional nebulizing chamber.

A conventional nebulizer is used to aerosolize liquid medication and deliver the aerosol for inhalation by a user. Although both are typically used for treating pulmonary medical conditions such as asthma, a conventional nebulizer differs from hand-held inhaler sprayers in that the hand-held aerosolizer generally contains multiple doses of medication, has a propellant permanently loaded within it, and is indicated for use where a single aerosolized dispensed quantity comprises the intended dose of medication for use by a patient.

It is critical to accurately time the dispensing shot from a hand-held medication inhaler to coincide with a user's inspiration, so as to ensure that the medication actually reaches the lungs of a user. Mistiming of the dispensing shot from a handheld inhaler can result in a short dose of medication or in no dose at all.

A conventional nebulizer, in contrast, has no stored medication at all. It is comprised of a nebulizing chamber, an air pump and an inhaler. The air pump, usually electrically driven, supplies a stream of compressed air through a conduit to a nebulizer housing. The housing is generally cylindrical, has a top and bottom part that can be separated by a user, and the top part has an upwardly projecting extension that ends in an inhaler. The inhaler is generally a horizontally disposed tube with an open distal end and a proximal end that is an open-ended mouthpiece.

The conventional nebulizer housing contains a nebulizing chamber. The chamber is basically a vertical cylinder with an open top for receiving a dose of liquid medication. The chamber has an air-stream inlet in the bottom. Compressed air from the air pump is conveyed to the chamber's bottom air inlet through a conduit. The compressed air enters the bottom of the nebulizing chamber and is then mixed with the dose of liquid medication, causing the medication to become nebulized into an aerosol. There is an open airflow between the nebulizer chamber and an upwardly extending short tube leading to a T-connection with a horizontal tube open at both ends that comprises an inhaler pipe with a breather mouthpiece at one end. One open end of the inhaler pipe comprises a distal end, opposite to a proximal end which comprises the mouthpiece shaped to fit into the mouth of a user.

The inhaler pipe is in open airflow with the nebulizer chamber. When a user inhales through the proximal open end of the mouthpiece, air is urged into the open distal end and into the proximal end of the mouthpiece. The user's inhalation effort also urges air from the nebulizer chamber, containing nebulized medication to rise up the connecting tube and to enter the proximal end of the mouthpiece.

The user thus inhales nebulized liquid medication, and the user may do so with inhalations repeated as needed over a period of time sufficient to get relief from respiratory symptoms that put the user into acute distress, such as an asthma attack. Thus an important difference between a conventional nebulizer and a hand-held inhaler is that the hand-held device is intended to deliver a single dose of medication intended to treat the entire episode of acute respiratory distress. The user must time the dispensing shot of the hand-held nebulizer to coincide with a breath inspiration or the effect of the device is defeated and the medication shot is wasted. In contrast, a conventional nebulizer provides the ability for an acute respiratory sufferer to breathe as many times as needed to receive sufficient nebulized medication into the lungs to alleviate the acute distress symptoms. The conventional nebulizer thus does a different job as compared to the hand held inhaler.

In additional comparison, handheld inhalers typically contain numerous doses of medication while a conventional nebulizer contains no medication at all.

A critical problem solved by the present invention is that, while medication delivered by a conventional nebulizer could be more effective than medication delivered by a hand-held inhaler due to the availability of repeated inhalations of medication with the conventional nebulizer, there remains an important shortcoming, which is addressed by the inventive step of the current invention.

In order to use a conventional nebulizer it is necessary for a user, or someone assisting the user to (1) disassemble the nebulizer housing by removing its top so as to expose the nebulizing chamber; (2) locate a separately stored container of liquid medication to be nebulized; (3) carefully open the liquid medication container so as not to spill it; (4) pour the liquid medication directly into the nebulizing chamber without losing any of it through spilling into the nebulizer housing; (5) reassemble the nebulizer housing; and (6) position the inhaler mouthpiece in the mouth so as to inhale the nebulized medication.

A problem arises in that use of a nebulizer is not going to be sought until a person is already in acute respiratory distress. Otherwise, problems of nebulizer overuse, overmedication, medication side effects and a search for alternate pulmonary therapy modalities will all become concerns for a patient. Therefore, use of a conventional nebulizer implies that a user is experiencing acute pulmonary symptoms, is in acute distress, and is experiencing an emergency.

Persons suffering acute respiratory distress are routinely subject to being fearful, frightened, or fully panicked. Fear, fright and panic are well known to degrade performance on tasks requiring some level of skill in eye-hand coordination tasks. When seeking the use of a conventional nebulizer, then, a user is required to locate a separate container holding a dose of liquid medication, open the nebulizer, open the medication container, pour the liquid into the nebulizer chamber, and re-assemble the nebulizer housing. The aforedescribed sequence of steps can be difficult or impossible for a fearful, frightened or panicked sufferer of acute respiratory distress. An important consideration is that there will almost certainly be occasions when a person experiencing acute need of a conventional nebulizer is alone and without anyone to assist. It is just these occasions where a conventional nebulizer may be available but be impossible for a user to operate.

To solve the problem of user inability to operate a conventional nebulizer in an emergency, the present invention presents a simple solution: construct a conventional nebulizer than has a built-in stored dose of liquid medication and make that liquid dose injectable into the nebulizer chamber with either a simple twist of a screw cap (preferred embodiment of the present invention) or a single stroke of user force (non-preferred embodiment). As provided in the present invention the user will not be required to disassemble or reassemble the housing of a conventional nebulizer; will not be required to locate a separately stored container of liquid medication; will not be required to open the separate medication container; and will not be required to pour the liquid medication into the nebulizer chamber.

According to the present invention, a conventional nebulizer will have added to its housing a storage chamber, preferably cylindrical, for storing, in loaded-gun fashion, a dose of liquid medication on board the conventional nebulizer housing. In the preferred embodiment of the present invention the novel storage chamber for the medication capsule is a substantially cylindrical sleeve with an open top aperture projecting vertically downward from the inhaler pipe to a point slightly above the conventional nebulizer chamber within the housing of a conventional nebulizer. The sleeve's diameter is small enough so as not to interfere with the conventional nebulizer's free flow of air from the nebulizer chamber, up the conventional neck of a nebulizer and into the conventional inhaler pipe of a nebulizer. The medication capsule storage sleeve merely occupies a portion of the air passage between the nebulizer chamber and the inhaler pipe and thus in no way does the storage sleeve seal or impede the conventional free flow of air within what is otherwise a conventional nebulizer.

In the non-preferred embodiment of the present invention the novel medication storage chamber (as with the preferred embodiment, the storage chamber of the non-preferred embodiment is also sleeve-like); however, instead of extending vertically as does the sleeve of the preferred embodiment, the storage chamber of the non-preferred embodiment generally projects outwardly from an inner delivery end in proximity to the nebulizing chamber, through the wall of a conventional nebulizer housing, and extends to an outer user-access end.

In the non-preferred embodiment the novel structure medication storage chamber generally has a tapered-nozzle open-aperture delivery end disposed in close proximity to the nebulizing chamber so that the liquid medication, when deployed by a user, is injected reliably and directly into the nebulizing chamber.

In the non-preferred embodiment (as is also true for the preferred embodiment) the novel medication storage chamber accepts a single disposable and user-replaceable cartridge containing a dose of liquid medication to be nebulized in an emergency. The chamber of the non-preferred embodiment is provided at its outer end with pressure means for a user to exert a stroke of physical force upon the outer end of the medication chamber so as to squeeze upon the medication cartridge. The cartridge of the non-preferred embodiment is preferably cylindrical with an inner end tapered to match and fit within the tapered nozzle of the medication storage chamber of the non-preferred embodiment. In the non-preferred embodiment an outer end of the medication cartridge is capable of accepting force from a manually-operated piston.

The medication dose cartridge of the non-preferred embodiment has a seal that is capable of rupture upon application of hydraulic pressure, the seal being located at an inner end of the cartridge, disposed at or near the inner end of the medication storage chamber. The preferable emergency user-pressure means is a piston arrangement, where the piston is integral with the medication cartridge, is elastomerically sealed, and accepts a push-force from a piston rod. The easily recognized example of this is a medical syringe.

The non-preferred embodiment of the present invention may be described as a medical syringe capable of receiving disposable medication dose cartridges, in combination with a conventional nebulizer. The piston arrangement (exemplified by a medical syringe) projects through the wall of the conventional nebulizer and has its medication delivery nozzle at or very near the conventional nebulizing chamber. Conventional stop means at the outer end of the chamber prevents the piston rod from coming out of the outer end of the medication storage chamber.

In another embodiment, insertion of the medication capsule within a rotatable knob cam activation assembly facilities bursting of the seal of the medication reservoir capsule.

The novel combination of the present invention addresses and solves the problem of what procedure must be followed by a patient having a breathing emergency, such as a severe attack of asthma, and needs a quick reliable dose of nebulized medication, particular where (1) no other person is available to assist the patient and (2) a single-shot hand-held nebulizer is medically inappropriate for treatment.

### DETAILED DESCRIPTION OF THE DRAWINGS

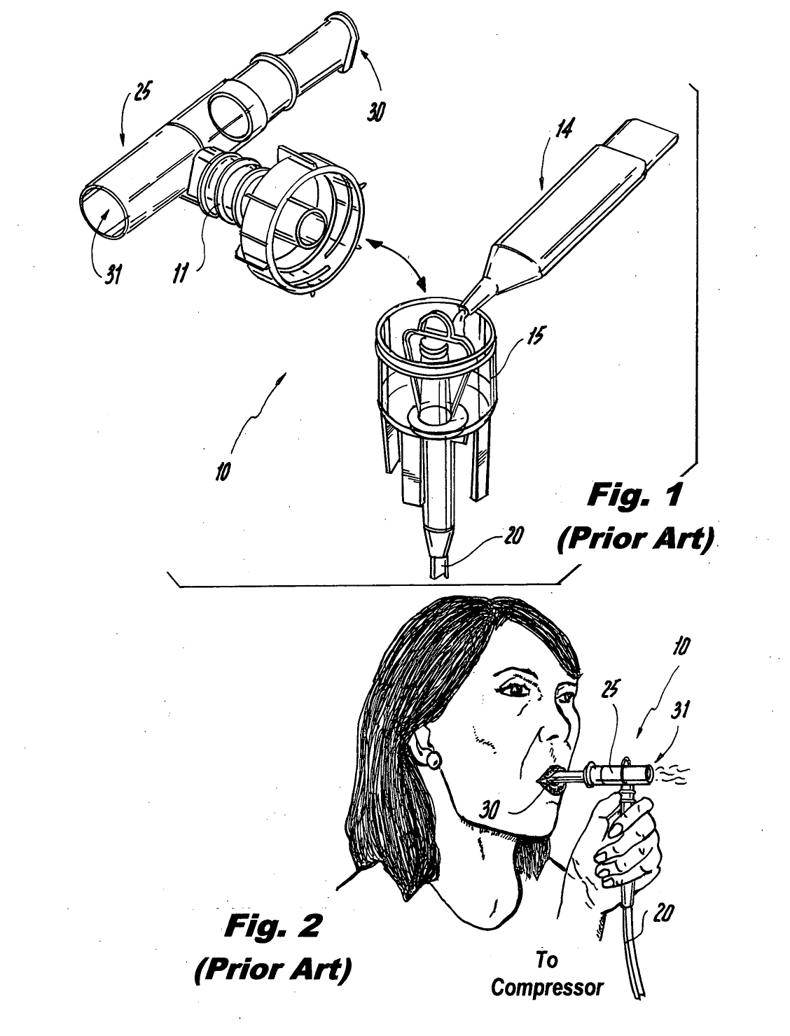

FIG. 1 is an exploded view of a prior art conventional nebulizer housing 10 shown disassembled. Conventional medication container 14 is shown adding liquid medication to conventional nebulizing chamber 15. In the event of a respiratory emergency, a user would have to locate a separate container of liquid medication 14, then open it, then disassemble (as shown) the portions of the nebulizer housing 10, then pour the liquid medication from its separate container 14 into nebulizer chamber 15, then reassemble nebulizer housing 10 before being able to inhale nebulized medication through proximal end of conventional mouthpiece 30 which is part of conventional breather 25, breather 25 having an open distal end 31 opposite to proximal end 30.

When a user has pour medication into nebulizer chamber 15 and reassembled housing 10, then conventional air supply line 20 supplies a stream of compressed air to nebulizer chamber 15 causing the liquid medication to become nebulized and urging the nebulized medication upward through connecting tube 11 so as to be available for user inhalation through proximal end mouthpiece 30.

FIG. 2 shows a perspective view of the prior art conventional nebulizer in use. A user inserts the proximal mouthpiece end 30 of breather 25 into the mouth and inhales. Nebulizer housing 10 [concealed by the user's hand in the drawing] furnishes nebulized (aerosolized) medication to the user for as many repeated inhalations as the user may need for alleviation of an acute respiratory emergency. Air supply lines 20 is shown extending upwardly but the user's hand conceals the intersection of air supply line 20 with the bottom of the nebulizer chamber 10.

In a non-preferred embodiment of the present invention, the novel medication storage chamber generally projects outwardly from an inner delivery end in proximity to the nebulizing chamber, through the wall of a conventional nebulizer housing, and extends to an outer user-access end.

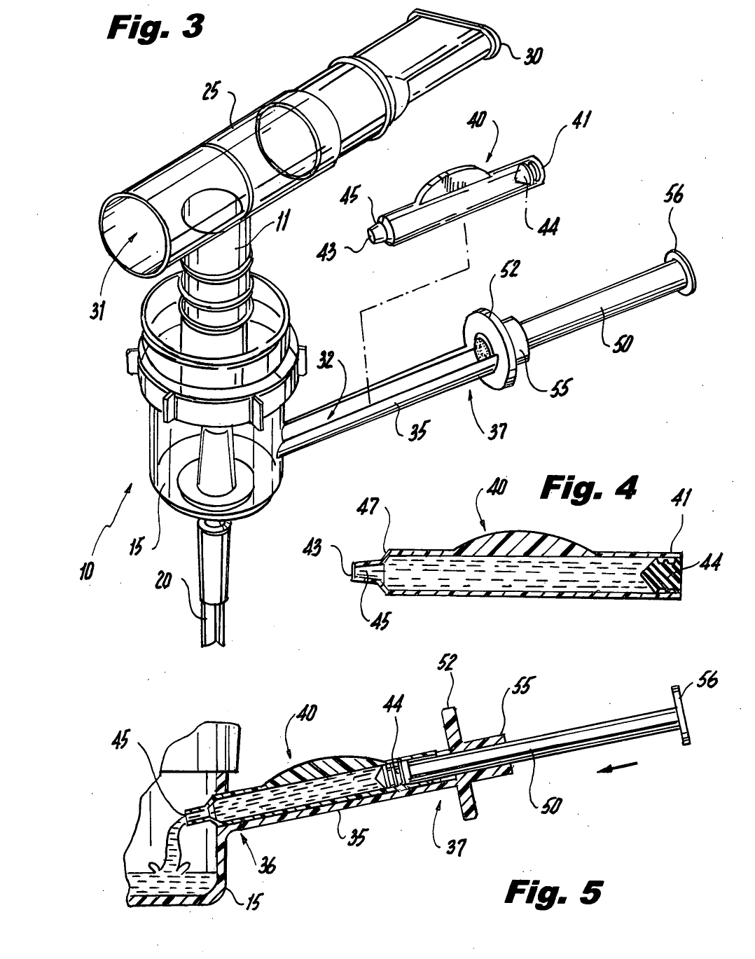

FIG. 3 shows the non-preferred embodiment of the present invention with a conventional nebulizer housing 10 fitted with novel integral (i.e., built-in) medication storage chamber 35. Storage chamber 35 is capable of receiving removable medication dose cartridge 40. Both chamber 35 and matching cartridge 40 are elongated, preferably cylindrical and both have matching inner and opposite outer ends. The inner end 36 of storage chamber 35 is disposed within nebulizer housing 10 while outer end 37 of chamber 35 is outside of nebulizer housing 10. Chamber 35 is fixed in a position that places its inner end 36 in close proximity to nebulizing chamber 15. The preferably cylindrical body of chamber 35 points radially outward from nebulizing chamber 15 so that outer end 37 of medication storage chamber 35 is outside of and spaced apart from nebulizer housing 10.

Medication storage chamber 35 is provided with open-ended tapered nozzle 38 at its inner end 36, nozzle 38 being in close proximity to nebulizing chamber 15 so as to reliably inject a dose of liquid medication from cartridge 40 upon user application of a single inwardly directed pressure stroke to pressure plate 56 of grooved piston rod 50, disposed within medication storage chamber 35, at the outer end 37 of said storage chamber.

Medication cartridge 40 is provided with tapered inner end 42 tapered to open end 45. Pressure seal 43 is located at inner end 42 of cartridge 40 while elastomerically sealed piston 44 is located at the outer end of cartridge 44. Upon user application of a single stroke of inward pressure on pressure plate 56 at the outer end of piston rod 50 (user grasps Finger Engagements Wings 52 for convenience), contact is made between grooved piston rod 50 and piston 44 resulting in an increase in hydraulic pressure on seal 43. Tapered shoulders 47 of cartridge 40 contact and engage tapered nozzle 38 of medication storage chamber 35, causing cartridge 40 to become seated firmly within cartridge 35 when a user applies manual pressure to pressure plate 56 of grooved piston rod 50.

Seal 43 is manufactured so as to burst upon user force application on pressure plate 56 of grooved piston rod 50. When seal 43 bursts, pressure from grooved piston rod 50 causes injection of liquid medication from cartridge 40 into nebulizing chamber 15. The remainder of the nebulizing operation is conventional.

FIG. 4 shows the non-preferred embodiment of the present invention with a detail of removable medication dose cartridge 40, having pressure seal 43 disposed at inner end 42, open end 45 is comprised of the tapered shoulders 47 at inner end 42 of cartridge 40 and outer end 41 contains movable elastomerically sealed piston 44. Piston 44 receives pressure from grooved piston rod 50. In response, piston 44 moves in an inward direction applying hydraulic pressure to the liquid medication contained within the body of cartridge 40. In turn the hydraulic pressure causes seal 43 at the inner end of cartridge 40 to burst. When seal 43 ruptures, liquid medication is forced under piston pressure to be injected into nebulizing chamber 15.

FIG. 5 shows the non-preferred embodiment of the present invention with a cut away side view detail of medication storage chamber 35 intersecting nebulizer housing 10 so as to have inner end 36 of chamber 35 in close proximity to nebulizing chamber 15 for reliable injection into chamber 15 of liquid medication from open inner end 43 of cartridge 40 upon application of a single stroke of inward user pressure upon pressure plate 56 of grooved piston rod 50, the force being transmitted to piston 44 of cartridge 40. Stop 55 engages groove on Piston Rod 50, preventing piston Rod 50 from coming out of medication storage chamber 35.

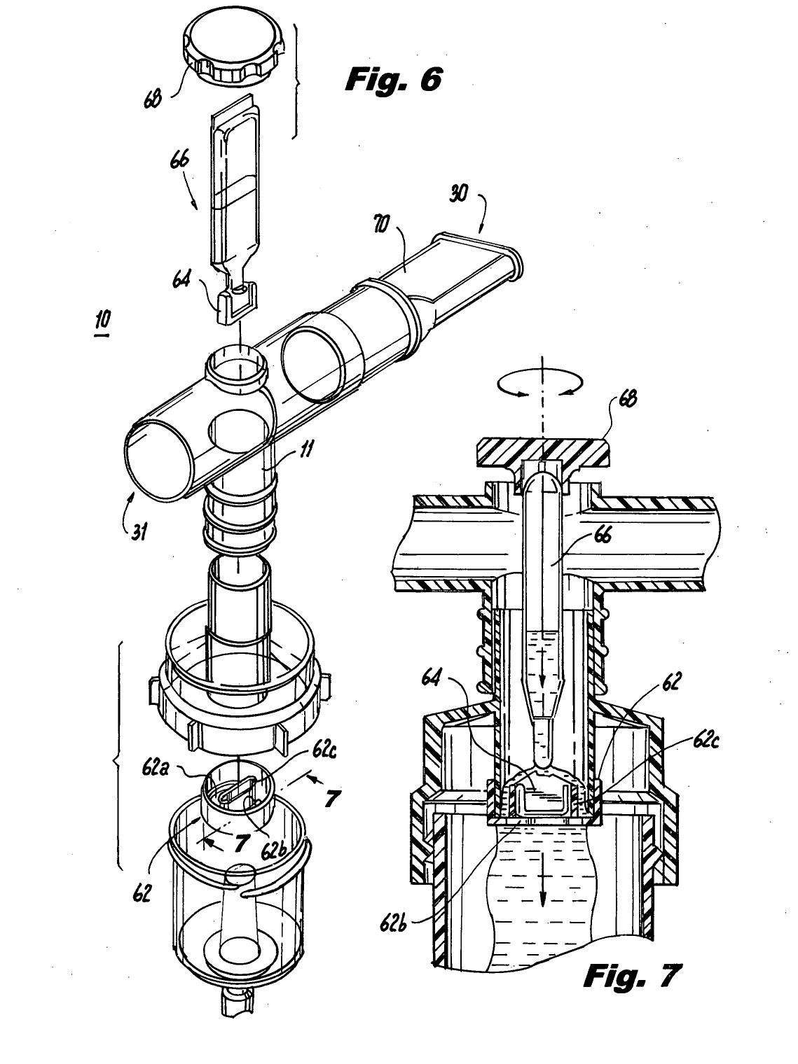

As shown on FIGS. 6 and 7, in a preferred embodiment, the novel medication storage sleeve 62 projects vertically downward from the top of horizontal inhaling pipe 70 extending downwardly into the nebulizer housing 10 to a point just above the nebulizing chamber 15. A medication dose capsule 66 is an elongated substantially cylindrical container oriented vertically within sleeve 62.

Capsule 66 is user inserted and user removed respectively to and from sleeve 62. Capsule 66 is intended to be stored in sleeve 66 until used, and then removed and replaced in preparation for a next use of the nebulizer.

Capsule 66 has a lower end tear off tab 64. Sleeve 62 has lower end stop means 62c to engage tear off tab 64 to prevent tab 64 from turning when torque is applied to capsule 66. Stop means 62a is attached by a retention means, such as bracket 62b, within hollow sleeve 62, allowing fluid flow of the liquid medication through lots 62a and 62b and then through aperture 62d of hollow sleeve 62.

Sleeve 62 accepts screw cap activating handle 68 after a user inserts capsule 66 into sleeve 62. Screw cap 68 engages projection means on capsule 66 so as to twist capsule 66 within sleeve 62 when a user applies a torque force to screw cap 68. Because the lower end tear off tab 64 of capsule 66 is prevented from twisting by the stop means 62a within sleeve 66, capsule 66 is caused to shear and rupture at its lower end when a user twists cap 68. Liquid medication within capsule 66 flows by gravity into nebulizing chamber 15 upon rupture of the lower end of capsule 66. The liquid medication is then conventionally nebulized and the user gets the therapeutic benefit of the nebulizer in a conventional manner.

FIG. 6 shows an exploded view of the preferred embodiment novel medication storage sleeve 62 projects vertically downward from the top of horizontal inhaling pipe 70 extending downwardly into the nebulizer housing 10 to a point just above the nebulizing chamber 15. A medication dose capsule 66 is an elongated substantially cylindrical container oriented vertically within sleeve 62.

Capsule 66 is user inserted and user removed respectively to and from sleeve 62. Capsule 66 is intended to be stored in sleeve 66 until used, and then removed and replaced in preparation for a next use of the nebulizer.

Capsule 66 has a lower end tear off tab 64. Sleeve 66 has lower end stop means to engage tear off tab 64 to prevent tab 64 from turning when torque is applied to capsule 66.

Sleeve 62 accepts screw cap activating handle 68 after a user inserts capsule 66 into sleeve 62. Screw cap 68 engages projection means on capsule 66 so as to twist capsule 66 within sleeve 62 when a user applies a torque force to screw cap 68. Because the lower end tear off tab 64 of capsule 66 is prevented from twisting by the stop means within sleeve 66, capsule 66 is caused to shear and rupture at its lower end when a user twists cap 68. Liquid medication within capsule 66 flows by gravity into nebulizing chamber 15 upon rupture of the lower end of capsule 66. The liquid medication is then conventionally nebulized and the user gets the therapeutic benefit of the nebulizer in a conventional manner.

FIG. 7 shows a detailed perspective of the preferred embodiment of the present invention. A user applies torque to screw cap 68 which in turn applies torque to medication capsule 66 seated within storage sleeve 62. Stop means 62c engages tear off tab 64 so that applied torque causes rupture of capsule 66, allowing its contents to flow by gravity into conventional nebulizer chamber 15.

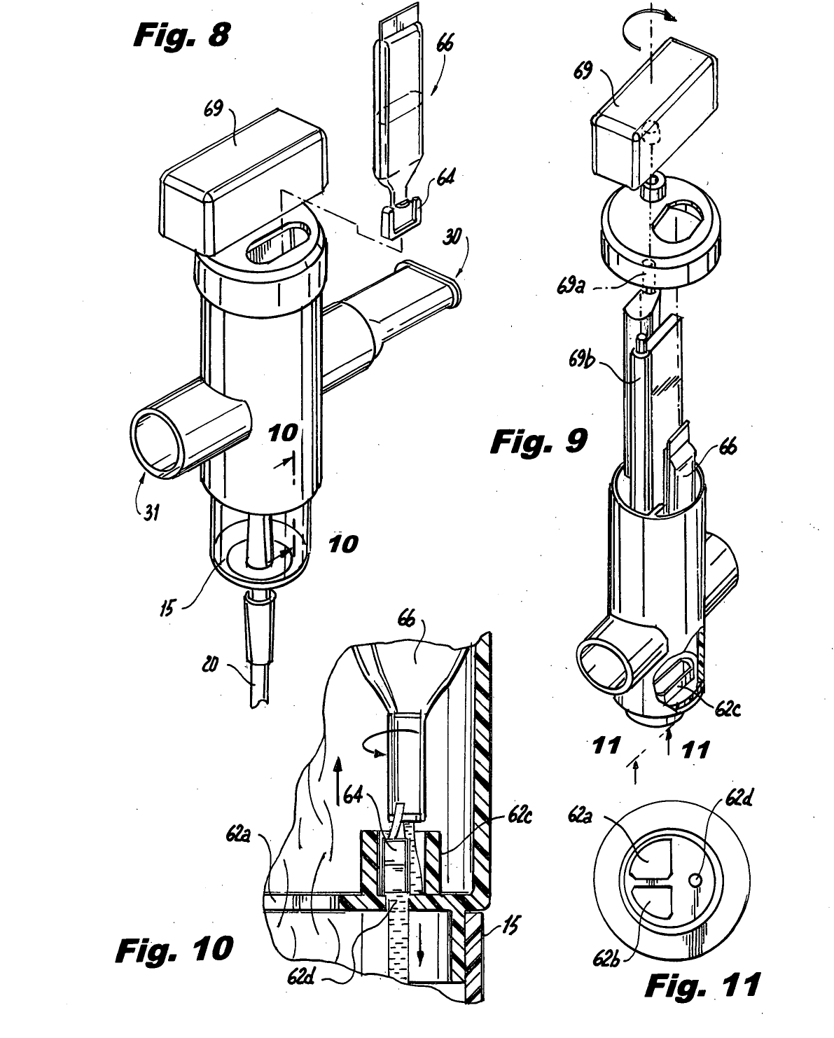

FIG. 8 shows the second version of the preferred embodiment, having a vertical storage sleeve 62 for a capsule 66 of liquid medication, where the capsule 66 is seated with its tear-off tab 64 in close proximity to the conventional nebulizing chamber within the housing of the conventional nebulizer.

FIGS. 9, 10 and 11 show a vertical storage sleeve 62 for the capsule 66 of liquid medication, showing a lever 69 actuating lever arm 69a, which exerts pressure against lever arm paddle 69b against capsule 66, thereby moving the capsule 66 laterally, while the tear-off portion 64 of the capsule is seated and immobilized within stop means 62c, so that lateral pushing of the capsule 66 causes a tear of the capsule 66 at the tear-off portion 64 and fluid flow through slots 62a and 62b adjacent to stop means 62c, through aperture 62d and into the fluid reservoir portion 15 of the nebulizer.

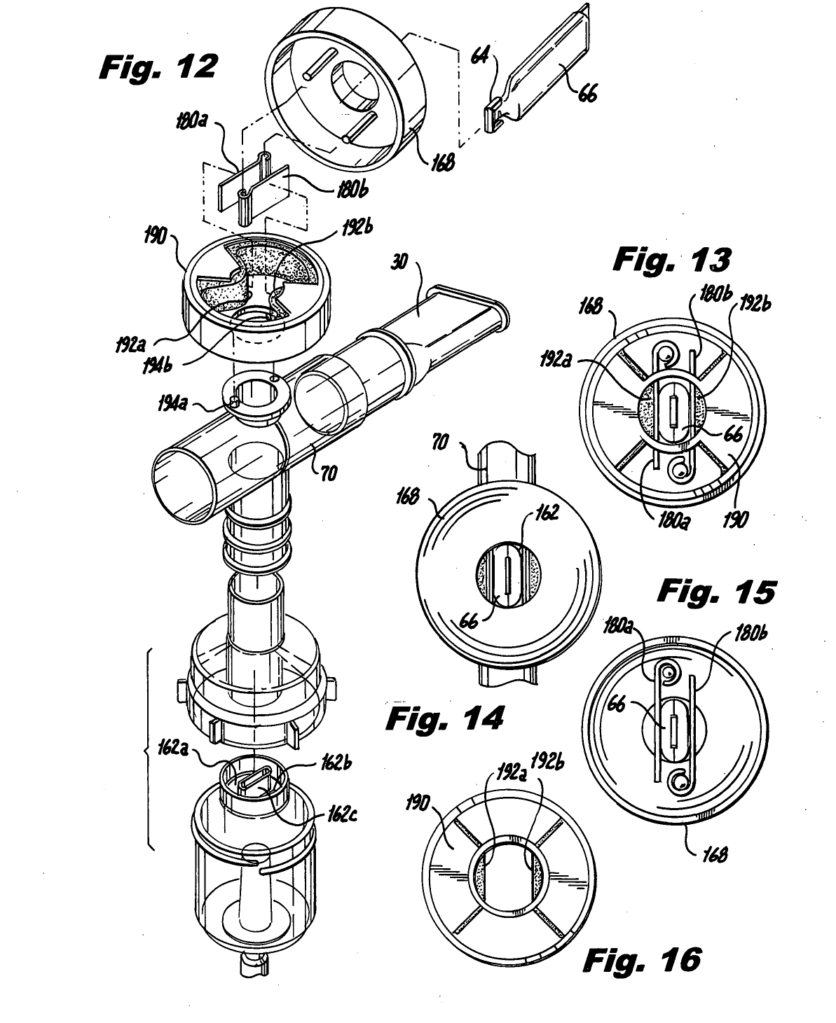

FIGS. 12-16 show an alternate embodiment for a knob cam assembly for bursting the tear off tab 64 from capsule 66. As shown in FIG. 12, capsule 66 is inserted through a port in knob activator 168 between capsule pincher blades 180a and 180b, down to restraining stop means 162c, adjacent to one or more mist ports 162a and/or 162b, etc., which, after bursting of the seal between capsule 66 and tear off tab 68, medication is misted within nebulizer 15 upward to inhaling pipe 70 and mouthpiece 30. Ports 162a and/or 162b, as well as restraining stop means 162c are down stream of inhaling pipe 70, between nebulizer 15 and inhaling pipe 70.

Rotation of knob activator 168 causes twisting of capsule 66 between capsule pincher blades 180a and 180b, and thence against cam contact protrusion elements 192a and 192b of cam assembly 190, which rotates in unison with rotation of knob activator 168, while restraining stop means holds tear off tab 64 of capsule 66 during rotation of capsule 66 within cam assembly 190.

Rotation of knob activator 168 and cam assembly 190 is limited to a preferable arc of movement, such as, for example 180 degrees, by means of reciprocating stop element 194a on inhalation pipe 70 being stopped by reciprocating stop element 194b on the adjacent bottom of cam assembly 190.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention, as noted in the appended Claims.

I claim:

1. A medication-nebulizer comprising a user-deployable emergency medication dose and means for deploying said emergency dose of medication embedded into and projecting outwardly from a conventional nebulizer housing having a conventional nebulizing chamber within said housing.

2. The medication-nebulizer of claim 1 wherein said means for deploying said emergency dose of medication comprises a tubular medication dose storage chamber having a pair of opposite ends, one of said ends being an inner nebulizer end fixed in close proximity to said conventional nebulizing chamber and wherein said opposite end is an outer end, said tubular storage chamber projecting radially outward from near said conventional nebulizing chamber within said conventional nebulizer housing through said conventional housing so that the outer end of said tubular storage chamber is conveniently accessible to a user.

3. The medication-nebulizer of claim 2 wherein said outer end of said tubular medication dose storage chamber is provided with cap means for convenient user cap removal and replacement, and wherein said tubular medication dose storage chamber removably receives a disposable cartridge containing an emergency dose of medication to be nebulized.

4. The medication-nebulizer of claim 3 wherein said disposable medication cartridge comprises an inner end having a pressure-burstable seal near said inner end, said inner end being stored within said tubular medication dose storage chamber wherein said inner end of said cartridge is nested near said inner end of said tubular medication dose storage chamber when said chamber is loaded with a stored cartridge containing an emergency medication dose.

5. The medication-nebulizer of claim 4 wherein said inner end of said tubular medication dose storage chamber comprises a tapered nozzle having an open end, said nozzle being in fixed position such that upon user emergency seal-burst deployment of the liquid medication out of said cartridge, said storage chamber nozzle aims a flow of said liquid medication so as to be reliably delivered directly into said conventional nebulizing chamber.

6. The medication-nebulizer of claim 5 wherein said outer end of said tubular medication dose storage chamber comprises means for applying pressure upon said outer end of said disposable medication cartridge, said outer end of said disposable medication cartridge comprising a deformable pressure-receiving means.

7. The medication-nebulizer of claim 6 wherein said means for applying pressure upon said outer end of said medication cartridge comprises piston means,

8. The medication-nebulizer of claim 7 wherein said piston means is integral with said disposable medication dose cartridge.

9. The medication-nebulizer of claim 8 wherein said disposable medication dose cartridge comprises a user removable, user replaceable medical syringe preloaded with an emergency dose of medication to be nebulized.

10. The medication-nebulizer of claim 9 wherein said means for deploying said emergency dose of medication comprises application of inwardly-directed user pressure upon said piston of said syringe cartridge, said pressure causing failure of said pressure-burst seal and injecting said liquid medication dose through said nozzle of said tubular medication dose storage chamber and into said conventional nebulizing chamber.

11. A method for speeding relief to sufferers of acute respiratory distress by reducing the time and effort required to inject liquid medication in a nebulizer comprising the steps of
1. applying a single stroke of force to a nebulizer for deploying a stored dose of liquid medication into the nebulizing chamber; and
2. inhaling the nebulized medication.

12. The method of claim 11 wherein said method employs a nebulizer having an integrally built-in storage chamber and delivery means for respectively storing a dose of liquid medication and for delivering said medication dose reliably to the nebulizer chamber in an emergency.

13. The method of claim 12 wherein the means for delivery of medication to the nebulizer chamber comprises a medication dose cartridge maintained within a storage chamber, said storage chamber being integral with said nebulizer, and wherein said medication dose cartridge has a pressure-burstable seal for bursting upon user application of hydraulic pressure by piston means.

14. A medication-nebulizer having a built-in storage sleeve for storing a conveniently user-insertable and alternately user-removable emergency medication dose capsule and means for delivering said emergency dose of medication to a conventional nebulizer chamber within a conventional nebulizer housing.

15. The medication-nebulizer of claim 14 wherein said means for delivering said emergency dose of medication comprises a screw cap inserted over a medication capsule stored vertically within said storage sleeve, wherein said medication capsule has a bottom tear-off tab for causing failure of said capsule upon application of torque to said screw cap by a user, in turn transmitting said torque to capsule; and wherein said sleeve has stop means near a bottom end thereof for engaging said tear-off tab and preventing it from turning upon application of torque to said capsule.

16. The medication-nebulizer of claim 15 wherein said bottom tear-off tab is located in close proximity to the conventional nebulizing chamber so as to permit gravity delivery of the contents of said capsule upon torque produced rupture of capsule.

17. A method for speeding relief to sufferers of acute respiratory distress by reducing the time and effort required to inject liquid medication in a nebulizer comprising the steps of
1. applying a single twist of torque force to a screw cap engaged with a medication capsule for delivering a stored dose of liquid medication into the nebulizing chamber; and
2. inhaling the nebulized medication.

18. The method of claim 17 wherein said method employs a nebulizer having an integrally built-in storage sleeve and delivery means for respectively storing a dose of liquid medication and for delivering said medication dose reliably to the nebulizer chamber in an emergency.

19. The method of injecting medication into a nebulizer as in claim 17 wherein said means for dispensing an emergency dose of medication comprises a knob cam assembly for bursting a tear off tab from a medication capsule; wherein said capsule is inserted through a port in a knob activator between respective capsule pincher blades, down to a capsule restraining stop means, adjacent to at least one respective mist port, wherein, after bursting of a seal between said capsule and said tear off tab, medication is misted within a nebulizer upward to an inhaling pipe and a mouthpiece for inhalation therefrom.

20. The method of injecting medication into a nebulizer as in claim 19 wherein said at least one mist-accommodating port and said capsule restraining stop means are located down stream of inhaling pipe, between the nebulizer and said inhaling pipe.

21. The method of injecting medication into a nebulizer as in claim 19 wherein said at least one mist accommodating port is a plurality of mist accommodating ports.

22. The method of injecting medication into a nebulizer of claim 19 wherein rotation of said knob activator causes twisting of capsule between capsule pincher blades, and thence against cam contact protrusion elements of said cam assembly, which said cam assembly rotates in unison with rotation of knob activator, while restraining stop means holds tear off tab of capsule during rotation of capsule within cam assembly.

23. The method of injecting medication into a nebulizer of claim 22 wherein rotation of said knob activator and said cam assembly is limited to a preferable arc of movement by means of a reciprocating stop element located on said inhalation pipe being stopped by a reciprocating stop element located on an adjacent bottom portion of said cam assembly.

24. The method of injecting medication into a nebulizer of claim 22 wherein rotation of said knob activator and said cam assembly is limited to an arc of movement of 180 degrees.

25. A medication-nebulizer comprising a user-deployable emergency medication dose and means for deploying said emergency dose of medication embedded into a conventional nebulizer housing having a conventional nebulizing chamber within said housing.

26. The medication nebulizer as in claim 25 wherein said means for dispensing an emergency dose of medication comprises a knob cam assembly for bursting a tear off tab from a medication capsule; wherein said capsule is inserted through a port in a knob activator between respective capsule pinchers, down to a capsule restraining stop means, adjacent to at least one respective mist port, wherein, after bursting of a seal between said capsule and said tear off tab, medication is misted within a nebulizer upward to an inhaling pipe and a mouthpiece for inhalation therefrom.

27. The medication nebulizer as in claim 26 wherein said at least one mist-accommodating port and said capsule restraining stop means are located down stream of inhaling pipe, between the nebulizer and said inhaling pipe.

28. The medication nebulizer as in claim 26 wherein said at least one mist accommodating port is a plurality of mist accommodating ports.

29. The medication nebulizer of claim 26 wherein rotation of said knob activator causes twisting of capsule between capsule pinchers, and thence against cam contact protrusion elements of said cam assembly, which said cam assembly rotates in unison with rotation of knob activator, while restraining stop means holds tear off tab of capsule during rotation of capsule within cam assembly.

30. The medication nebulizer of claim 29 wherein rotation of said knob activator and said cam assembly is limited to a preferable arc of movement by means of a reciprocating stop element located on said inhalation pipe being stopped by a reciprocating stop element located on an adjacent bottom portion of said cam assembly.

31. The medication nebulizer of claim 25 wherein said means comprises a vertical storage sleeve for the capsule of liquid medication, at least one lever actuating a paddle exerting lateral pressure against the capsule within said storage sleeve, thereby moving the capsule laterally, while a tear-off portion of the capsule is seated and immobilized within a stop, thereby causing a tear of the capsule at the tear-off portion thereof and fluid flow into a fluid reservoir portion of the nebulizer.
